(19) United States
(12) Patent Application Publication
(10) Pub. No.: US 2020/0129735 A1
(43) Pub. Date: Apr. 30, 2020

(54) TRANSPARENT CATHETER AND HUB

(71) Applicant: HeartStitch, Inc., Fountain Valley, CA (US)

(72) Inventors: Anthony Nobles, Fountain Valley, CA (US); Dru Dobbs, Fountain Valley, CA (US)

(21) Appl. No.: 16/664,633

(22) Filed: Oct. 25, 2019

Related U.S. Application Data
(60) Provisional application No. 62/750,754, filed on Oct. 25, 2018.

(51) Int. Cl. A61M 25/00

(52) U.S. Cl. CPC A61M 25/0097

(57) ABSTRACT

A catheter assembly comprises a transparent hub and a transparent catheter coupled to the transparent hub at a proximal end. The present inventive concept further contemplates that the transparent hub can further comprise components, including but not limited to, a valve, a valve housing, a catheter securing mechanism, a hub body. The present inventive concept further contemplates a catheter assembly comprising one or more transparent components, such that the catheter assembly reveals the contents therein to a user.

This application claims the benefit of and priority to U.S. Provisional Patent Application No. 62/750,754, filed Oct. 25, 2018, which is incorporated by reference herein in its entirety.

### FIELD OF THE INVENTION

The field of the invention is catheter systems.

### BACKGROUND

Conventional catheter systems do not allow the internal contents of the catheter to be viewed from the outside, primarily because they use opaque or substantially opaque materials that significantly restrict the ability of a user to ascertain the contents of the catheter. For example, the catheter portions of many existing catheter systems are made of opaque materials. Similarly, the hub portions of many existing catheter systems are also entirely made of, or substantially comprise, opaque materials.

U.S. Pat. No. 5,188,596 to Condon discloses a transparent prostate dilation balloon and scope. However, Condon contemplates the use of a catheter that is partially or completely transparent to position balloons for prostate dilation. However, Condon does not contemplate the use of a transparent catheter assembly that can also include a catheter hub or additional components (e.g., side line).

U.S. Pat. No. 6,942,648 to Schaible similarly discloses a transparent intraluminal catheter. Similarly to Condon, however, Schaible does not contemplate the use of a transparent catheter assembly which can also include a catheter hub and additional components (e.g., side line).

Condon, Schaible, and all other extrinsic materials discussed herein are incorporated by reference to the same extent as if each individual extrinsic material was specifically and individually indicated to be incorporated by reference. Where a definition or use of a term in an incorporated reference is inconsistent or contrary to the definition of that term provided herein, the definition of that term provided herein applies and the definition of that term in the reference does not apply.

Transparent catheter systems comprising multiple subcomponents that are partially or completely transparent would address a significant problem in existing catheter systems. By allowing a user to see inside each component of the catheter assembly, certain procedures can be executed more efficiently and life-threatening medical issues can be avoided.

For example, many procedures use catheters to introduce medical instruments that are directed through the catheter to a target location to complete a procedure (e.g., vascular surgery). Such procedure can require a surgeon to engage in guesswork to fix a malfunction. A transparent catheter and hub assembly would allow the surgeon to quickly identify the malfunction, and correct it without having to remove the instrument, or try to correct the issue at the expense of patient safety.

In another example, catheters can be used to introduce medicines directly into the bloodstream. However, air can also be introduced into the bloodstream through catheter systems, which can cause air or gas embolisms if not removed. Transparent catheter and hub assemblies allow medical professionals to directly check whether there is air in the line, and then take corrective action to reduce stroke and heart attack, or other potentially fatal conditions caused by air embolisms.

Thus, there is a need for apparatus, methods, and systems that use transparent catheters and transparent hubs to solve existing issues in otherwise conventional catheter systems.

### SUMMARY OF THE INVENTION

Among other things, the inventive subject matter provides a catheter assembly comprising a substantially transparent hub and a substantially transparent catheter.

It is further contemplated that the inventive concept herein can include many variations and compositions comprising substantially transparent and substantially non-transparent materials. In some embodiments, different compositions of substantially transparent materials can be used for their advantageous physical and/or optical properties. For example, substantially transparent materials can comprise uniformly distributed mixtures or layers of different materials.

The inventive subject matter further contemplates that the substantially transparent hub comprises the components of a valve, a valve housing, a hub body, and a securing mechanism, each of which is substantially transparent.

### BRIEF DESCRIPTION OF THE DRAWINGS

FIG. 1 depicts an embodiment of a substantially transparent catheter and hub assembly.

FIG. 2A depicts a substantially transparent catheter hub and substantially transparent valve housing.

FIG. 2B depicts a substantially transparent catheter securing mechanism.

FIG. 2C depicts a substantially transparent catheter.

### DETAILED DESCRIPTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

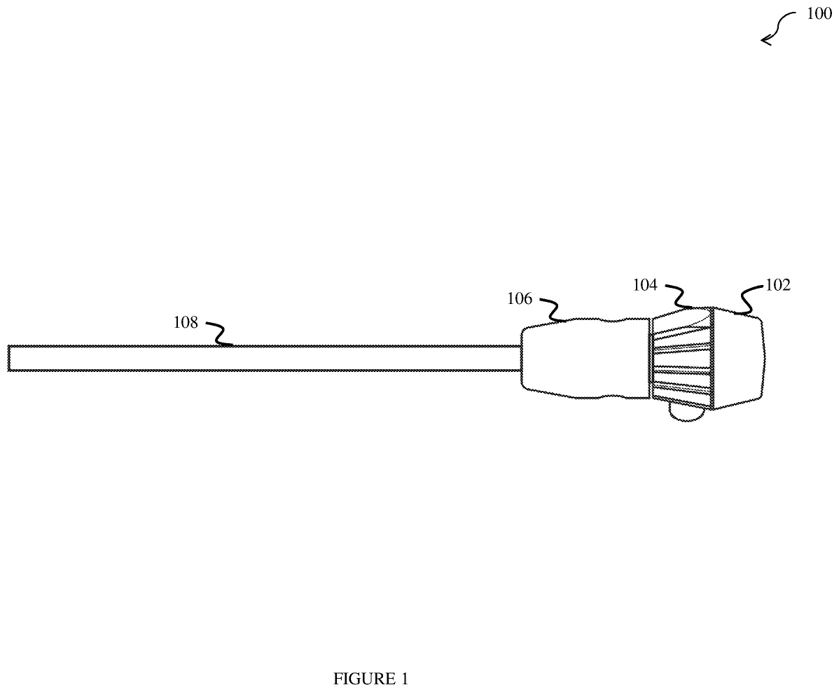

FIG. 1 depicts an embodiment of a catheter and hub assembly. Catheter and hub assembly 100 comprises valve housing 102, hub body 104, securing mechanism 106, and catheter 108.

In catheter and hub assembly 100, a distal end of valve housing 102 couples to a proximal end of hub body 104. The distal end of hub body 104 couples to a proximal end of securing mechanism 106. Catheter 108 is frictionally secured to catheter and hub assembly 100 using pressure exerted on catheter 108 by coupling hub body 104 and securing mechanism 106.

It is contemplated that catheter and hub assembly 100 can comprise any combination of the depicted components in any configuration. The individual components comprising catheter and hub assembly 100 are discussed in more detail below.

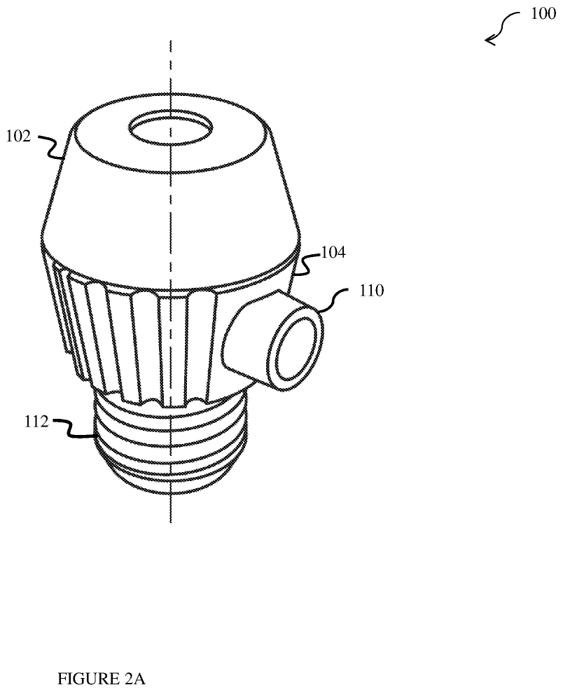

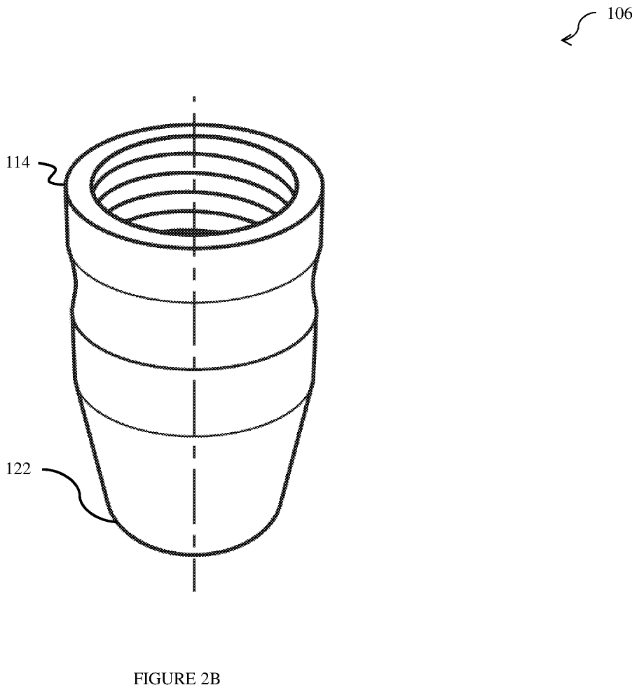

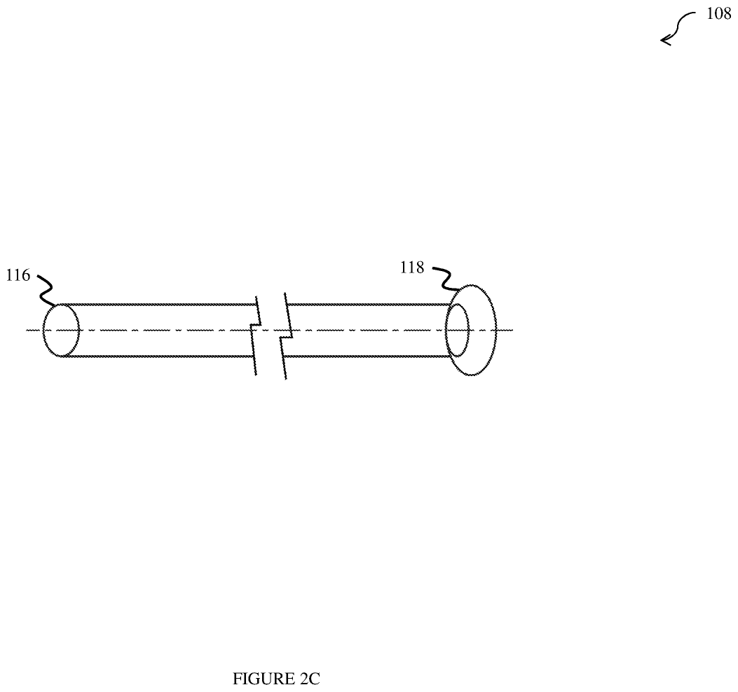

FIG. 2A depicts a substantially transparent valve housing 102 and substantially transparent hub body 104.

Valve housing 102 includes valve 120 disposed within. In a preferred embodiment, it is contemplated that valve housing 102 comprises a proximal aperture to allow one or more instruments to access a lumen of valve housing 102 through valve 120 and a distal aperture to couple with a proximal end of hub body 104. The distal aperture is contemplated to have a coupling means to couple valve housing 102 to hub body 104.

It is contemplated that valve 120 can comprise any valve known in the art. In the depicted embodiment, valve 120 comprises a silicon valve that comprises a slit to allow an instrument to access the lumen of hub body 104 through valve 120.

In other embodiments, valve 120 can be mechanically actuated. For example, valve 120 can be a spring-based mechanism actuated using pressure to create a path through the hub body. In another embodiment, valve 120 can be actuated using the elastomeric properties of a material. For example, valve 120 can be a one-way valve that opens when an object in inserted but is sealed shut upon exposure to back pressure (e.g., back pressure from a blood vessel).

Hub body 104 comprises side port 110 and coupling mechanism 112. Hub body 104 defines a lumen that is accessible through valve 120 and side port 110.

Side port 110 is configured to allow access to the lumen of hub body 104. For example, a tube can be inserted through the side port to deposit medicine directly into the bloodstream of a patient. In alternative embodiments, hub body 104 does not include side port 110. In yet another alternative embodiment, hub body 104 can include multiple side ports 110.

In preferred embodiments, side port 110 is substantially transparent. In alternative embodiments, side port 110 can be a substantially non-transparent material. For example, side port 110 can be made of surgical grade steel. In another example, side port 110 can be made of an opaque plastic material.

Coupling mechanism 112 comprises any means of removably coupling with securing mechanism 106.

In a preferred embodiment, coupling mechanism 112 is threaded to mate with a corresponding threaded object. However, it is contemplated that coupling mechanism 112 can use any type of securing mechanism. For example, coupling mechanism 112 can use an elastomeric material to removably seal coupling mechanism 112 to a corresponding structure. In a second example, coupling mechanism 112 can be a snap-fit mechanism. In a third example, coupling mechanism 112 can be a magnetic mechanism. In a fourth example, coupling mechanism 112 can be a locking mechanism, such as a twist-lock mechanism.

It is further contemplated that the embodiments and examples associated with coupling mechanism 112 can be applied to all components that couple to other components disclosed herein.

As used herein, “substantially transparent” should be interpreted as inclusive of any material that allows ambient visible light to pass through, to an intensity that is readily seen by a human eye. In preferred embodiments, substantially transparent is defined as any physical property of a material through which a user can use such light to distinguish between blood and air bubbles. It is contemplated what is considered “substantially transparent” can change based on one or more of the functionality of the device, the physical properties of the material, the physical properties of a device comprising the material (e.g., thickness of device components), the composition of a second material to be viewed through the substantially transparent material (e.g., high contrast v. low contrast), and the environment.

It is contemplated that substantially transparent materials includes homogenous transparent materials and heterogeneous transparent materials. For example, a homogeneous transparent material can be entirely made of clear polyethylene (PE). In another example, a homogeneous material can comprise a mixture of clear PE and clear polyethylene terephthalate (PTE).

It also is contemplated that the inventive concept herein can comprise any suitable substantially transparent material or materials. For example, the substantially transparent materials can comprise one or more of plastic materials (e.g., polypropylene, polyethylene, nylon, PVC or PTFE), metal materials (e.g., aluminum, copper, platinum, metal alloys, etc.), silicon, glass fiber, or any suitable combination thereof.

In some embodiments, substantially transparent materials are a biocompatible material. In other embodiments, substantially transparent materials can comprise one or more of organic fiber-based materials, bioplastics, and biodegradable plastics.

Bioplastics can include, but are not limited to, polyhydroxylalkanoates (PHAs), polylactic acid (PLA), thermoplastic polymers (e.g., starch blends), and cellulose-based plastics.

In another example, heterogeneous transparent materials can comprise multiple types of materials, wherein at least one of the materials is substantially transparent. For example, a heterogeneous transparent material can comprise an opaque material with a clear glass window. In another example, a heterogeneous transparent material can comprise multiple transparent components made of distinct materials that have been combined (e.g., layered).

For example, valve housing 102 can be substantially transparent for blood work if it includes a translucent material that allows a doctor to see through valve housing 102 to detect any bubbles forming in the blood inside. In another example, valve housing 102 can be substantially transparent for a suprapubic catheter it includes a nearly transparent material that allows a doctor to see through valve housing 102 to detect the presence of blood in the urine.

In some situations, a material can be substantially transparent for certain applications but not for others. For example, the material can be a transparent material with optical filtering properties, such as colored glass, to increase the contrast between particular colors when seen through the glass. In another example, a catheter material may be substantially transparent for detecting blood in urine, but, the material may not be substantially transparent for detecting bubbles forming in blood. However, this example is merely illustrative and contemplates the use of any materials that have exhibit different optical properties in different situations.

As defined herein, the “distal” used with respect to catheters should be interpreted as towards the end of the catheter that opens into the patient, and “proximal” should be interpreted as towards the opposite end.

FIG. 2B depicts a substantially transparent catheter securing mechanism 106 with proximal aperture 114 and distal aperture 122.

Catheter securing mechanism 106 comprises proximal aperture 114 at a first end and distal aperture 122 at a second end, which defines a through-hole. In a preferred embodiment, proximal aperture 114 is threaded to receive complementary threading from coupling mechanism 112. However, it is contemplated that proximal aperture 114 and coupling mechanism 112 can removably couple using any method known in the art. For example, proximal aperture 114 and coupling mechanism 112 can couple using a friction fit.

Proximal aperture 114 and distal aperture 122 are adapted to receive catheter 108, discussed in further detail below.

FIG. 2C depicts a catheter 108 with distal end 116 and proximal end 118.

Catheter 108 can be any catheter known in the art. For example, catheter 108 can be a straight catheter. In another example, catheter 108 can be a coude catheter. In yet another example, catheter 108 can be a balloon catheter.

It is further contemplated that substantially transparent catheter 108 can alternatively be substantially non-transparent in some embodiments.

As depicted, proximal end 118 is flared. In preferred embodiments, distal end 116 is inserted through proximal aperture 114 and distal aperture 122 of catheter securing mechanism 106. The flared shape of proximal end 118 prevents proximal end 118 from exiting through distal aperture 122.

It is contemplated that proximal end 118 is pinched between hub body 104 and securing mechanism 106 to create a flare fitting when hub body 104, securing mechanism 106, and catheter 108 are used in conjunction.

In some embodiments, proximal end 118 is not flared and can be secured using any mechanism known in the art. For example, proximal end 118 can be secured using a friction fit. In another example, proximal end 118 can be secured using an adhesive. In yet another example, proximal end 118 and catheter securing mechanism 106 can be one continuous structure.

Distal end 116 can be adapted to receive any one or more attachments using any attachment mechanism known in the art. For example, substantially transparent catheter 108 can be a balloon catheter.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A catheter assembly, comprising:
a substantially transparent hub; and
a substantially transparent catheter coupled to the substantially transparent hub at a proximal end.

2. The catheter assembly of claim 1, wherein the substantially transparent hub further comprises:
a valve;
a valve housing, wherein the valve housing is removably coupled to the valve;
a catheter securing mechanism; and
a hub body,
wherein the hub body couples to the valve housing at the proximal end,
wherein the hub body couples to the catheter securing mechanism at a distal end, and
wherein coupling the hub body to the catheter secures the substantially transparent catheter to the hub body.

3. The catheter assembly of claim 2, wherein the valve housing removably couples to the hub body.

4. The catheter assembly of claim 2, wherein the catheter securing mechanism removably couples to the hub body.

5. The catheter assembly of claim 2, wherein the hub body and the catheter securing mechanism are fixably coupled.

6. The catheter assembly of claim 2, wherein the hub body and the valve housing are rotatably coupled using a threaded fastener-based mechanism.

7. The catheter assembly of claim 2, wherein the hub body and the valve housing are fixably coupled.

8. The catheter assembly of claim 2, wherein the valve housing further comprises a valve port.

9. The catheter assembly of claim 2, wherein the hub body further comprises a side port.

10. The catheter assembly of claim 1, wherein the substantially transparent hub and the substantially transparent catheter comprises one or more polymeric materials.

11. The catheter assembly of claim 10, wherein the one or more polymeric materials are selected from the group consisting of: polyethylene terephthalate (PTE), polyethylene (PE), polyvinyl chloride (PVC), polypropylene (PP), polystyrene (PS), polylactic acid (PLA), polycarbonate (PC), acrylic (PMMA), polyoxymethylene (POM), nylon (PA), polyurethane (PU), polyetheretherketone (PEEK), and acrylonitrile butadiene styrene (ABS).

12. The catheter assembly of claim 10, wherein the hub body and the catheter securing mechanism are removably coupled using a threaded fastener-based mechanism.

13. The catheter assembly of claim 12, wherein coupling the hub body and the catheter securing mechanism using the threaded fastener-based mechanism exerts pressure on a portion of the substantially transparent catheter to pinch the substantially transparent catheter between the hub body and the catheter securing mechanism.

14. The catheter assembly of claim 1, wherein the substantially transparent catheter is removably coupled to the substantially transparent hub.
